(19) United States
(12) Patent Application Publication
(10) Pub. No.: US 2010/0281765 A1
(43) Pub. Date: Nov. 11, 2010

(54) EFFICIENT BIOMASS FRACTIONATING SYSTEM FOR AN ENERGY PULSE CROP

(76) Inventor: Anne M. Schwartz, Tualatin, OR (US)

Correspondence Address: Mark Farrell Patent Law, Inc., PC, P.O. Box 142056, Spokane, WA, 99214, US

(21) Appl. No.: 12/774,702

(22) Filed: May 5, 2010

Related U.S. Application Data
(60) Provisional application No. 61/175,788, filed on May 5, 2009.

(51) Int. Cl. C10L 1/182; A23L 1/28; A23L 1/00; C12P 7/06

(52) U.S. Cl. 44/451; 426/425; 426/52; 435/161

(57) ABSTRACT

An efficient biomass fractionating system for an energy pulse crop is provided. Pulses include, e.g., peas, beans, and lentils. A method and ecosystem model applies a premium utilization to each fraction of a pulse crop so that no fraction is treated as waste. The methods may also be applied to other alternative crops, such as chestnut seeds, banana and 408 peel, and taro root. One example method removes a protein fraction first, as a food source, before using the remaining fractions to produce energy products, such as ethanol or methane, increasing the efficiency of the entire fractionating process. The fractionating method enables an ecosystem, in which pulses grow inexpensively on low-grade land or under poor conditions providing a cash crop food, energy, and chemical components. In a farm co-op model, the pulse crop provides sustainability as participants inexpensively produce protein, ethanol, and industrial chemical components.

### RELATED APPLICATIONS

This patent application claims priority to U.S. Provisional Patent Application No. 61/175,788 to Schwartz, filed May 5, 2009, and incorporated herein by reference in its entirety.

### BACKGROUND

Conventional corn to ethanol or grass and wood biomass operations require a great deal of pesticides or herbicides, nitrogen fertilizers, and water. For example, production of ethanol from corn is up to six times less efficient than producing ethanol from sugarcane. Ethanol production from corn is highly dependent upon subsidies and it consumes a primary food crop to produce fuel.

Subsidies paid to fuel blenders and ethanol refineries have often been cited as the reason for driving up the price of corn, and in farmers planting more corn and converting considerable land to corn (maize) production which generally consumes more fertilizers and pesticides than many other land uses. This is at odds with the subsidies actually paid directly to farmers that are designed to take corn land out of production and pay farmers to plant grass and idle the land, often in conjunction with soil conservation programs, in an attempt to boost corn prices. Grass biomass is often high in ash and bio-oil made from grasses contains substances that convert to tar and gum up turbines within 8 months of operation. Conventional ways of converting a biomass to ethanol are wrought with pitfalls.

### SUMMARY

An efficient biomass fractionating system for an energy pulse crop is provided. Pulses include, e.g., peas, beans, and lentils. A method and ecosystem model applies a premium utilization to each fraction of a pulse crop so that no fraction is treated as waste. The methods may also be applied to other alternative crops, such as chestnut seeds, banana and peel, and taro root. One example method removes a protein fraction first, as a food source, before using the remaining fractions to produce energy products, such as ethanol or methane, increasing the efficiency of the entire fractionating process. The fractionating method enables an ecosystem, in which pulses grow inexpensively on low-grade land or under poor conditions providing a cash crop food, energy, and chemical components. In a farm co-op model, the pulse crop provides sustainability as participants inexpensively produce protein, ethanol, and industrial chemical components.

### BRIEF DESCRIPTION OF THE DRAWINGS

FIG. 1 is a diagram of an example fractionation of a pulse crop.

FIG. 2 is a diagram of an example self-sustaining economic model based on a pulse crop fractionation.

FIG. 3 is a block diagram of an example pulse fractionating system.

FIG. 4 is a flow diagram of an example method of fractionating a pulse crop.

### DETAILED DESCRIPTION

### Overview

This disclosure describes an efficient biomass fractionating system for pulse crops (e.g., legumes) targeted to provide a cost effective model for energy production, whereby for example, a food grade protein concentrate is produced which covers a cost of feedstock, then leftover material, such as starch and fiber are processed into energy. The exemplary fractionating system uses pulse crops as a framework, and treats each fraction of the fractionated pulse crop in a premium utilization, such that no fraction is considered a waste to be dealt with. The methods may also be applied to other alternative crops, such as chestnut seeds, banana and peel, and taro root.

“Pulses” as used herein, include the seedpod of leguminous plants (such as peas, beans, and lentils) or the leguminous plants themselves (i.e., “legumes”). Conventional methods typically seek an energy product from a grain (e.g., ethanol from a corn crop) and then deal with leftover byproducts of the process as best they can. See, for example, U.S. patent application Ser. No. 11/077,971 to Lewis—the “Lewis reference”—entitled “Continuous process for producing ethanol using raw starch,” which is incorporated herein by reference in its entirety.

In contrast to conventional techniques, the example methods described herein remove one or more fractions of the crop first, usually as valuable food components, before producing an energy component such as an alcohol. Such a technique achieves a premium utilization of each fraction of the pulse crop, rather than allowing the utilization of one fraction (e.g., energy production) to degrade the utilization of another fraction of the pulse crop. One result is that pulse crops, one of the most inexpensive crops to grow and among the hardiest crops to grow on low-grade land or under poor conditions, becomes a valuable crop that can produce food, energy, and chemical components—and on land that might otherwise go unused or discarded. The example methods described herein enable pulse crops to become a viable and valuable asset (money-making crop) in many parts of the world.

A unique pulse processing model solves economic and environmental problems associated with energy crops and energy production since pulse crops do not require nitrogen or pesticides. Besides not polluting water with run-off fertilizer, pulse crops require less gasoline to produce the crop, an estimated savings of five gallons per acre, because tractors are not required to apply fertilizer and pesticide substances on crops. Also some pulses, such as lentils can be grown on 50% of CRP qualified lands netting an additional 50% profit from the amount farmers are paid to leave these lands fallow. Using this model: a) farmers are sustained by higher income per acre and increased demand, b) energy production of crops does not displace lands used for food production c) the crops prevent erosion to a higher degree than leaving the land fallow, d) the crops preserve the environment by not polluting it with nitrogen and pesticides require much less water, e) the cost of energy is low because the production of protein concentrate and a high protein extruded cereal covers the cost of the commodity, f) the purified starch is high quality, low ash and therefore coverts to energy at a higher percentage, and g) a government may save money by not having to pay farmers to leave the land fallow.

Pulse Crop Ecosystem

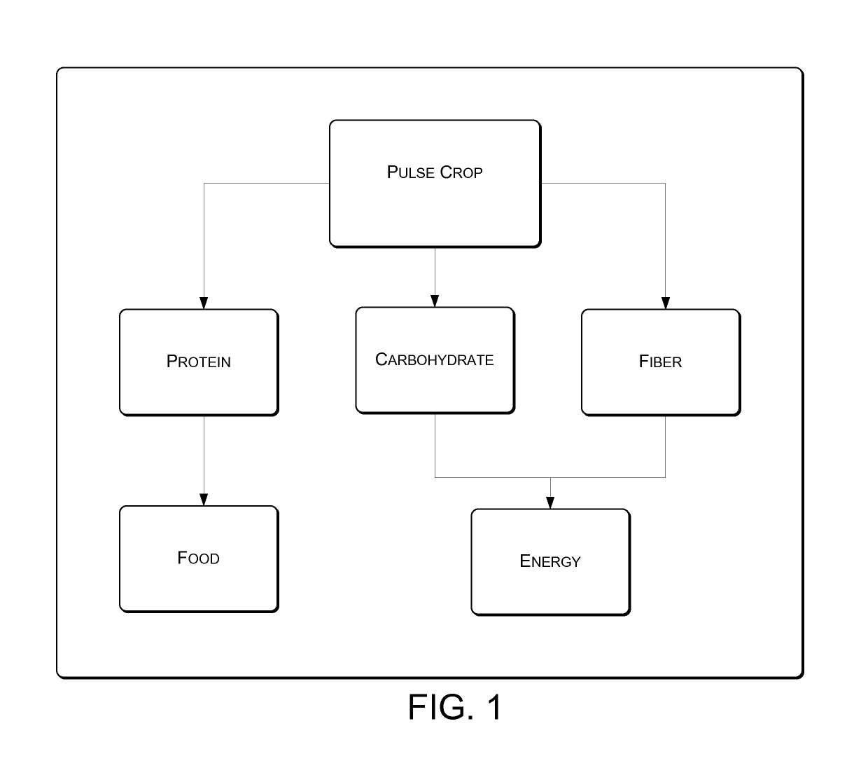

FIG. 1 shows an example fractionation of a pulse crop. An exemplary process uses a pulse crop, fractionates the pulse into fiber, protein, carbohydrate,—e.g., removes the protein fraction first, for food use—and then uses the carbohydrate primarily for making biofuel but in some cases also for making other byproducts, such as an edible gluten-free cereal (e.g., which can be optionally monitored for the presence of pesticides). The fiber and excess starch fraction may be used for making the biofuel. The protein fraction may be used to make a protein concentrate which can be used in a manner similar to how soy is currently used. Additionally the protein fraction can be included in a cereal formulation to produce a high protein and or gluten free cereal for human consumption, but in another implementation is primarily used for making a fish or animal feed.

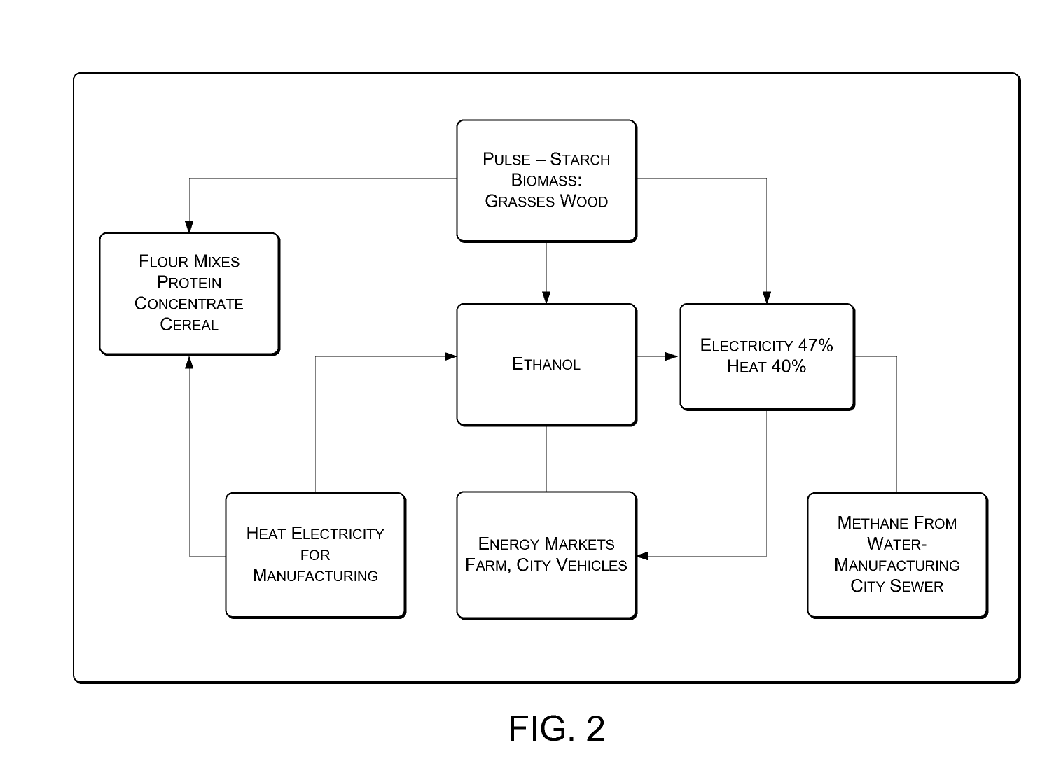

FIG. 2 shows an example self-sustaining economic model based on a pulse crop fractionation. In one implementation, the fractionating system provides a complete biomass solution for producing a protein product and ethanol and/or electricity: that is, an ethanol production plant with an ability to break down cellulose as well as utilize the starch in a legume crop. The system presents several favorable solutions to current problems, for example, a fish feed byproduct for aquaculture.

Aquaculture is currently seeing many different species of fish on the brink of extinction if the use of conventional fish meal continues as it has been over past years. The world demand for fish is increasing, while wild fish are in decreasing supply. Conventional fish feed is composed of a formulation that includes 50% fish meal because producers have not been able to replace this component for a number of different reasons. The example fractionating system enables a plant-based fish feed that is healthy for fish using 100% plant-based feed. Implemented by a farmer utilizing a pulse crop, the system can be implemented to increase the pulse crop by 40% using existing land: e.g., 50% of which can be utilized from CRP land (conservation reserve program land), and the remainder from fallow land.

The exemplary fractionating system is profitable to farmers, especially those in financial danger because of competition. Some countries have bred varieties of pulses that grow well in their various sub-climates, thus out-competing farmers in other countries. The exemplary system provides sustainability, through providing a cash crop to be used for making ethanol. Thus, the system provides sustainability for farmers, and sustainability for the land itself. Conventionally, when prices go down so much that it is not worth planting pulse crops, problems arise, because pulses are often the rotational crops for wheat, to regenerate the land by fixing nitrogen in the soil. Without the rotational pulse crop, more fertilizers are applied to the ground, with nitrogen in a form that runs off into watersheds and rivers. So, the exemplary fractionating system provides sustainability for land, and improvement of water resources.

The example system presents a complete solution, in the sense that the ethanol production increases profits for farmers, and aims to use the entire biomass of the pulse crop, processing the whole pulse plant, e.g., stems too, into ethanol: what used to be tilled under now gets turned to profit. The pulse crop becomes a sustainable, reproducible, and highly utilized product.

An example pulse crop ecosystem can generate a reproducible business model. In one scenario, a portable method of producing and utilizing a pulse crop is modeled after a “Minnesota co-op” system, which is similar to corn ethanol plants, that is, production frameworks to be run by farmer co-ops. Workers and service people can be trained to a particular standard set of equipment systems, procedures, and layouts. The exemplary fractionating system is reproducible for farmers in co-ops, for example, in legume growing states of the U.S. The system can help to sustain the farmers, provides increased profit, can enables farmers to directly use their own lentils, or peas, to produce ethanol much more inexpensively than from corn because the pulse crops do not require the same amounts of pesticides, water, and nitrogen fertilizers that corn requires, yet can provide a higher yield.

### Example System

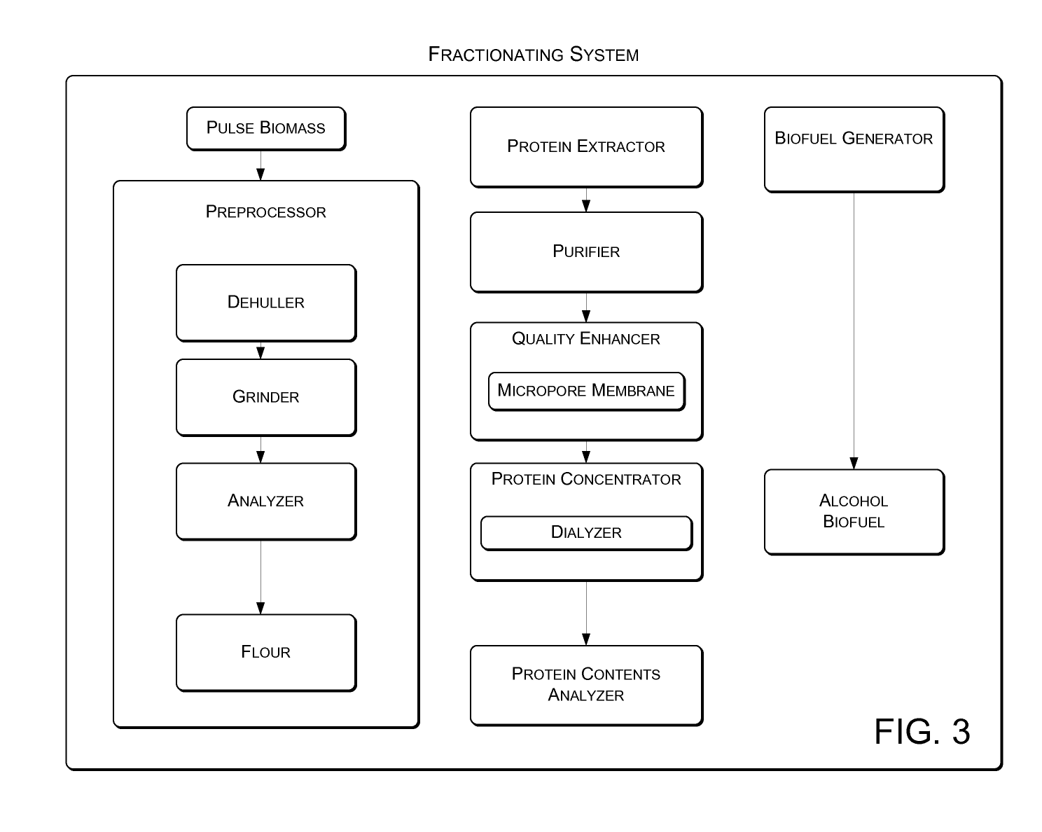

FIG. 3 shows an example pulse fractionating system. As one part of the pulse fractionating system, the biofuel generating part, a process such as that described in the above-cited Lewis reference is applied to a pulse crop instead of a grain crop, with innovative adjustments made to some of the steps and reagents in order to be compatible with, and to optimize, ethanol yields from pulses instead of grains.

In one implementation, for the fractionation of pulse seed, chestnut seed, banana and peel and taro root prior to using the process to make continuous alcohol or intermediates for plastic manufacturing the following is a pre-process:

Pre-Processing: Milling and Analysis of Legume Flour

In one implementation, legume seeds are dehulled using a tangential or other abrasive dehulling device to remove tannins and insoluble fibers abundant in the legume seed coat (TADD, Venables Machine Work, Ltd., Saskatoon, Canada). Dehulled seeds are then ground, e.g., with a CYCLONE or other similar mill fitted with a screen having, e.g., 0.1-0.4 mm openings (Udy Co., Fort Collins, Colo.). Pulse flours or other alternative crops mentioned above can be analyzed for moisture utilizing electronic sensors, for protein content (Method 46-30, AACC 2000), and for ash (Method 08-03, AACC 2000). Drying can occur automatically if moisture content is too high.

Pulse seeds or the other seeds, fruits or roots can also be milled to flour or mash using a device, such as a CHOPIN Experimental mill for production of a large quantity for protein extraction (Tripple & Renaud Co.). This mill is a type of roller mill, designed to mill wheat and also applicable to milling legumes. The mill effectively removes seed coats during milling process and produces a clean flour.

Pre-Processing: Extraction, Concentration and Isolation of Legume Seed Proteins

Wet fractionation methods with various salt concentrations, pH, and temperature levels are utilized to extract legume protein. The protein content of the extracts is between 40% and 100%.

Chickpea, lentil, pea, bean, chestnut, dried banana, and taro root are selected for processing and prepared to flour for wet fractionation.

Protein extractions may be conducted using three different extraction solutions, distilled water, a salt solution (0.5M NaCl) (Abdel-Aal et al 1986), and an alkaline solution (pH 9, 1.0N NaOH) (Tian et al 1999) at various temperature conditions.

The protein concentrates can be further purified to produce globulin isolates through the acid precipitation at various pH levels ranging from between approximately pH 3 and pH 6. An appropriate pH and other purification conditions may be selected based on yield and purity of globulin isolates.

Pre-Processing: Protein Quality Enhancement

Protein concentrates and isolates may be put through an electro-chemical filter utilizing an aluminum micro porous membrane to remove bioactive compounds, such as protein anti-nutritional or immune triggering proteins in pulse protein concentrates and isolates if desired or needed depending on the product that is to be manufactured from the concentrate.

Pre-Processing: Adjusting Protein Compositions of Concentrates and Isolates

Protein concentrates and globulin isolates of chickpea, pea (green, dried, or split) beans and lentils can be further fractionated to albumins and globulins by dialysis methods (e.g., at pH 4.6, with a 25 mM sodium citrate buffer).

Protein contents of albumin and globulin fractions can be determined by calculating the ratio of albumins to globulins. Albumin can be hydrolyzed and added back to the concentrate since albumin is not digested in its native form.

Biofuel Generation

In one implementation, an example method employs steps similar to those of the Lewis reference. The example method can produce ethanol from a pulse crop or other alternative crop (e.g., fractionated plant material). This method may include grinding the plant material (e.g., fractionated plant material) to produce ground plant material (e.g., fractionated plant material) including starch; saccharifying the starch, without cooking; fermenting the incubated starch; adding substrate continuously or clarifying continuously during fermentation; and recovering the ethanol from the fermentation. The present method can include varying the temperature during fermentation. The present method can include employing plant material (e.g., fractionated plant material) with a particle size such that more than 50% of the material fits though a sieve with a 0.5 mm mesh. The present method can yield a composition including at least 18% by volume ethanol. The process may includes producing starch from lentils and ethanol from the starch; producing dryer stack emissions including a significantly lower level of volatile organic compounds than conventional technologies since the protein is removed first.

The example process is capable of achieving extremely high relative ethanol % yield from a pulse crop when operating under a continuous clarification and continuous substrate addition mode of operation.

“Without cooking” may refer to a process for converting starch to ethanol without heat treatment for gelatinization and dextrinization of starch using alpha-amylase. Generally, for the processing a pulse crop, “without cooking” refers to maintaining a temperature below starch gelatinization temperatures, so that saccharification occurs directly from the raw native insoluble starch to soluble glucose while bypassing conventional starch gelatinization conditions. Starch gelatinization temperatures are typically in a range of 57 degrees C. to 93 degrees C. depending on the starch source and polymer type. In the example method, dextrinization of starch using conventional liquefaction techniques is not necessary for efficient fermentation of the carbohydrate in the pulse seed, legume, or other organic part.

“Plant material” refers to all or part of any plant (e.g., pulse seed or stem), typically a material including starch. Suitable plant material includes pulses (peas, e.g., whole ground peas), chickpeas, lentils, beans, chestnuts, taro root and bananas. The plant material can be a mixture of such materials and byproducts of such materials, e.g., pea fiber, stover, or other cellulose and hemicelluloses-containing materials such as wood or plant residues. Suitable plant materials for the example methods include pulses, chestnuts, taro root and bananas.

“Fractionated plant material” as used herein refers to plant material that includes only a portion or fraction of the total plant material, typically a material including starch. Fractionated plant material can include fractionated pulses such as fractionated peas (fractionated dry peas or split peas and including stems leaves and roots), fractionated lentils, including stems leaves and roots, fractionated chickpeas including stems leaves and roots, fractionated beans including stems leaves and roots, fractionated bananas including stems leaves and banana peels, fractionated chestnuts including stems and leaves, and fractionated starchy root crops, tubers, or roots such as fractionated taro root. Suitable fractionated plant materials include fractionated peas, either fractionated dry peas or fractionated split peas, lentils, chickpeas, beans, bananas, taro root and chestnuts.

As used herein, the terms “saccharification” and “saccharifying” refer to the process of converting starch to smaller polysaccharides and eventually to monosaccharides, such as glucose. Conventional saccharification uses liquefaction of gelatinized starch to create soluble dextrinized substrate which glucoamylase enzyme hydrolyzes to glucose. In an example method, saccharification refers to converting raw starch to glucose with enzymes, e.g., glucoamylase and acid fungal amylase (AFAU). According to one example method, the raw starch is not subjected to conventional liquefaction and gelatinization to create a conventional dextrinized substrate.

As described herein, a unit of acid fungal amylase activity (AFAU) refers to the standard Novozymes units for measuring acid fungal amylase activity. The Novozymes units are described in a Novozymes technical bulletin SOP No.: EB-SM-0259.02/01. Such units can be measured by detecting products of starch degradation by iodine titration. One unit is defined as the amount of enzyme that degrades 5.260 mg starch dry matter per hour under standard conditions.

A unit of glucoamylase activity (GAU) refers to the standard Novozymes units for measuring glucoamylase activity. The Novozymes units and assays for determining glucoamylase activity are described in a publicly available Novozymes technical bulletin.

A unit of amyloglucosidase activity (AGU) refers to the standard Novozymes units for measuring amyloglucosidase activity. The Novozymes units are described in a Novozymes technical bulletin SOP No.: EB-SM-0131.02/01. Such units can be measured by detecting conversion of maltose to glucose. The glucose can be determined using the glucose dehydrogenase reaction. One unit is defined as the amount of enzyme that catalyzes the conversion of 1 mmol maltose per minute under the given conditions.

As used herein, the terms “about” or “approximately” when used to modify an amount refers to the variation in that amount encountered in real world conditions of producing sugars and ethanol, e.g., in the lab, pilot plant, or production facility. For example, an amount of an ingredient employed in a mixture when modified by “about” includes the variation and degree of care typically employed in measuring in an ethanol production plant or lab. For example, the amount of a component of a product when modified by “about” includes the variation between batches in an ethanol production plant or lab and the variation inherent in the analytical method. Whether or not modified by “about,” the amounts include equivalents to those amounts. Any quantity stated herein and modified by “about” can also be employed as the amount: not modified by “about.”

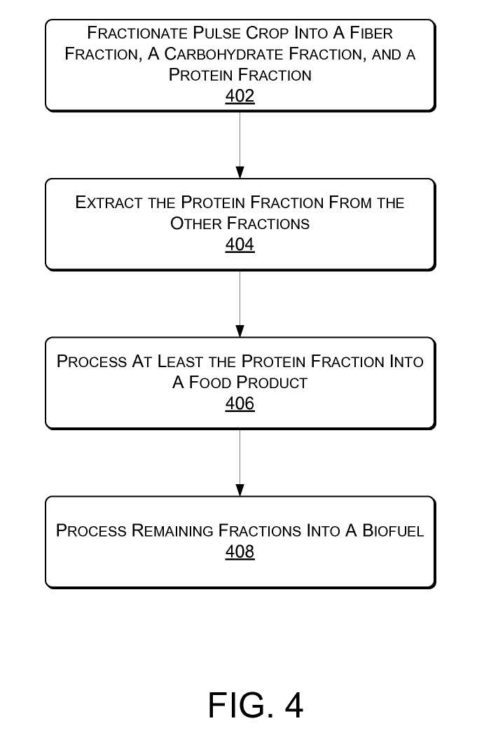

FIG. 4 shows an example method of fractionating a pulse crop. The steps are shown in individual blocks.

At block 402, a pulse is fractionated into a fiber fraction, a protein fraction, and a carbohydrate fraction.

At block 404, a protein fraction is extracted from the other fractions.

At block 406, at least the protein fraction is processed into a food product.

At block 408, after extracting the protein fraction, at least the carbohydrate fraction is processed into an alcohol biofuel.

Converting Pulse Starches to Ethanol

An example method produces high levels of alcohol during fermentation of pulse plant material (e.g., fractionated plant material). The method can include adding substrate continuously or clarifying continuously.

The present method converts starch from plant material (e.g., fractionated pulse plant material) to alcohol, such as ethanol. In an embodiment, the example method includes preparing the plant material (e.g., fractionated plant material) for saccharification, converting the prepared plant material (e.g., fractionated plant material) to sugars without cooking, and fermenting the sugars.

The plant material (e.g., fractionated pulse plant material) can be prepared for saccharification by any a variety of methods, e.g., by grinding, to make the starch available for saccharification and fermentation. In an embodiment, the vegetable material can be ground so that a substantial portion, e.g., a majority, of the ground material fits through a sieve with a 0.1-0.5 mm screen. For example, in an embodiment, about 70% or more, of the ground vegetable material can fit through a sieve with a 0.1-0.5 mm screen. In an embodiment, the reduced plant material (e.g., fractionated plant material) can be mixed with liquid at about 20 to about 50 wt-% or about 25 to about 45 wt-% dry reduced plant material (e.g., fractionated plant material).

The present process can include converting reduced plant material (e.g., fractionated plant material) to sugars that can be fermented by a microorganism such as yeast. This conversion can be effected by saccharifying the reduced plant material (e.g., fractionated plant material) with an enzyme preparation, such as a saccharifying enzyme composition. A saccharifying enzyme composition can include any of a variety of known enzymes suitable for converting reduced plant material (e.g., fractionated plant material) to fermentable sugars, such as amylases (e.g., alpha-amylase and/or glucoamylase). In an embodiment, saccharification is conducted at a pH of about 6.0 or less, for example, about 4.5 to about 5.0, for example, about 4.5 to about 4.8.

In an embodiment, the present method can include varying the pH. For example, fermentation can include filling the fermenter at pH of about 3 to about 4.5 during the first half of fill and at a pH of about 4.5 to about 6 (e.g., about 4.5 to about 4.8) during the second half of the fermenter fill cycle. In an embodiment, fermentation is conducted at a temperature of about 25 to about 40 degrees C., or about 30 to about 35 degrees C. In an embodiment, during fermentation the temperature is decreased from about 40 degrees C. to about 30 degrees C., or about 25 degree C., or from about 35 degrees C. to about 30 degrees C., during the first half of the fermentation, and the temperature is held at the lower temperature for the second half of the fermentation. In an embodiment, fermentation is conducted for about 25 (e.g., 24) to about to 150 hours, for example, for about 48 (e.g., 47) to about 96 hours.

The example method can include simultaneously converting reduced plant material (e.g., fractionated plant material) to sugars and fermenting those sugars with a microorganism such as yeast.

Ethanol can be recovered from the fermentation mixture by any of a variety of known processes, such as by distilling. The remaining stillage includes both liquid and solid material. The liquid and solid can be separated by, for example, centrifugation.

Preparing the Pulse or Plant Material

The plant material (e.g., fractionated pulse plant material) can be reduced by a variety of methods, e.g., by grinding, to make the starch available for saccharification and fermentation. Other methods of plant material reduction are available. For example, vegetable material, such as a pulse crop, can be ground with a ball mill, a roller mill, a hammer mill, or another mill known for grinding vegetable material, and/or other materials for the purposes of particle size reduction. The use of emulsion technology, rotary pulsation, and other means of particle size reduction can be employed to increase surface area of plant material (e.g., fractionated plant material) while raising the effectiveness of flowing the liquefied media. The prepared plant material (e.g., fractionated plant material) can be referred to as being or including “raw starch.”

A fine grind exposes more surface area of the plant material (e.g., fractionated pulse plant material), or vegetable material, and can facilitate saccharification and fermentation. In an embodiment, the vegetable material is ground so that a substantial portion, e.g., a majority, of the ground material fits through a sieve with a 0.1-0.5 mm screen. In an embodiment, about 35% or more of the ground material can fit through a sieve with a 0.1-0.5 mm screen. In an embodiment, about 35 to about 70% of the ground vegetable material can fit through a sieve with a 0.1-0.5 mm screen. In an embodiment, about 50% or more of the ground plant material can fit through a sieve with a 0.1-0.5 mm screen. In an embodiment, about 90% of the ground plant material can fit through a sieve with a 0.1-0.5 mm screen. In an embodiment, all of the ground plant material can fit through a sieve with a 0.1-0.5 mm screen. In an embodiment, the ground material has an average particle size of about 0.25 mm.

Plant Material Reduction

Preparing the plant material (e.g., fractionated pulse material) can employ any of a variety of techniques for plant material reduction. For example, the example method of preparing plant material can employ emulsion technology, rotary pulsation, sonication, magnetostriction, ferromagnetic materials, or the like. These methods of plant material reduction can be employed for substrate pretreatment. These methods can increase surface area of plant material (e.g., fractionated pulse material) while raising the effectiveness of flowing liquefied media (i.e., decreasing viscosity). These methods can include electrical to mechanical, mechanical to electrical, pulsation, and sound-based vibrations at varying speeds. These can provide varying frequencies over a wide range of frequencies, which can be effective for pre-treating the plant material (e.g., fractionated plant material) and/or reducing particle size.

Although not limiting to the present invention, certain of the sonic methods may create low pressure around a particle of plant material (e.g., fractionated pulse material) and induce cavitation of the particle or disruption of the particle structure. The cavitated or disrupted particle can increase availability of plant material (e.g., starch) to an enzyme, for example, by increasing surface area. It is believed that such pretreatment can decrease quantity of enzyme rates in the present method for ethanol production.

In an embodiment, the example method includes vibrating plant material (e.g., fractionated pulse material) and cavitating the fluid containing the pulse material. This can result in disrupting the plant material and/or decreasing the size of the plant material (e.g., fractionated plant material). In certain embodiments, the present method includes treating plant material (e.g., fractionated plant material) with emulsion technology, with rotary pulsation, with magnetostriction, or with ferromagnetic materials. This can result in disrupting the plant material and/or decreasing the size of the plant material (e.g., fractionated plant material). In an embodiment, the present method includes sonicating the plant material. This can result in disrupting the plant material and/or decreasing the size of the plant material (e.g., fractionated plant material).

The sound waves can be ultrasound. The present method can include sonicating the plant material at a frequency (e.g., measured in kHz), power (e.g., measured in watts), and for a time effective to reduce (or to assist in reducing) the particle size to sizes described above. For example, the method can include sonicating the plant material (e.g., fractionated plant material) at 20,000 Hz and up to about 3000 Watts for a sufficient time and at a suitable temperature. Such sonicating can be carried out with commercially available apparatus, such as high powered ultrasonics available from ETREMA (Ames, Iowa).

In an embodiment, the present method can include employing rotary pulsation for reducing plant material (e.g., fractionated plant material). The method can include rotary pulsating the plant material (e.g., fractionated plant material) at a frequency (e.g., measured in Hz), power (e.g., measured in watts), and for a time effective to reduce (or to assist in reducing) the particle size to sizes described hereinabove. Such rotary pulsating can be carried out with known apparatus, such as apparatus described in U.S. Pat. No. 6,648,500, the disclosure of which is incorporated herein by reference.

In an embodiment, the present method can include employing pulse wave technology for reducing plant material (e.g., fractionated plant material). The method can include rotary pulsing the plant material at a frequency (e.g., measured in Hz), power (e.g., measured in watts), and for a time effective to reduce (or to assist in reducing) the particle size to sizes described hereinabove. Such pulsing can be carried out with known apparatus, such as apparatus described in U.S. Pat. No. 6,726,133, the disclosure of which is incorporated herein by reference.

Fractionation

In the example method, the pulse material is fractionated into one or more components. For example, a material such as a pulse or lentil can be fractionated into components such as fiber (e.g., lentil fiber), germ (e.g., lentil germ), and a mixture of starch or fiber and protein to create a protein concentrate with from 45 to 100% purity (e.g., a mixture of pulse starch and pulse protein). One component or a mixture of these components can be fermented in a process according to the present invention. Fractionation of peas or another plant material can be accomplished by any of a variety of methods or apparatus. For example, a system manufactured by Satake can be used to fractionate plant material such as peas.

In one implementation, the germ and fiber components of the plant material can be fractionated and separated from the remaining portion of the vegetable material. In another embodiment, the remaining portion of the plant material (e.g., pea, lentil, bean, chickpea, chestnut, taro root and chestnut endosperm) can be further milled and reduced in particle size and then combined with the larger pieces of the fractioned germ and fiber components for fermenting.

In an embodiment, the plant material can be milled to access value added products (such as neutraceuticals, leutein, carotenoids, xanthophils, pectin, cellulose, lignin, mannose, xylose, arabinose, galactose, galacturonic acid, GABA, corn oil, albumins, globulins, prolamins, gluetelins, zein and the like).

Saccharification and Fermentation

The present process can include converting reduced plant material (e.g., fractionated plant material) to sugars that can be fermented by a microorganism such as yeast. This conversion can be effected by saccharifying the reduced plant material (e.g., fractionated plant material) with any of a variety of known saccharifying enzyme compositions. In an embodiment, the saccharifying enzyme composition includes an amylase, such as an alpha amylase (e.g., an acid fungal amylase). The enzyme preparation can also include glucoamylase. The enzyme preparation need not, and, in an embodiment, does not include protease. However, ethanol production methods according to the present invention can conserve water by reusing process waters (backset) which may contain protease. In an embodiment, the present method employs acid fungal amylase for hydrolyzing raw starch.

Saccharifying can be conducted without cooking. For example, saccharifying can be conducted by mixing source of saccharifying enzyme composition (e.g., commercial enzyme), yeast, and fermentation ingredients with ground pulses and process waters without cooking.

In an embodiment, saccharifying can include mixing the reduced plant material (e.g., fractionated plant material) with a liquid, which can form a slurry or suspension and adding saccharifying enzyme composition to the liquid. In an embodiment, the method includes mixing the reduced plant material (e.g., fractionated plant material) and liquid and then adding the saccharifying enzyme composition. Alternatively, adding enzyme composition can precede or occur simultaneously with mixing.

In an embodiment, the reduced plant material (e.g., fractionated plant material) can be mixed with liquid at about 20 to about 50 wt-%, about 25 to about 45 (e.g., 44) wt-%, about 30 to about 40 (e.g., 39) wt-%, or about 35 wt-% dry reduced plant material (e.g., fractionated plant material). As used herein, wt-% of reduced plant material in a liquid refers to the percentage of dry substance reduced plant material or dry solids. In an embodiment, the method of the present invention can convert raw or native starch (e.g., in dry reduced plant material) to ethanol at a faster rate at higher dry solids levels compared to conventional saccharification with cooking. Although not limiting to the present invention, it is believed that the present method can be practiced at higher dry solids levels because, unlike the conventional process, it does not include gelatinization, which increases viscosity.

Suitable liquids include water and a mixture of water and process waters, such as stillage (backset), scrubber water, evaporator condensate or distillate, side stripper water from distillation, or other ethanol plant process waters. In an embodiment, the liquid includes water. In an embodiment, the liquid includes water in a mixture with about 1 to about 70 vol-% stillage, about 15 to about 60 vol-% stillage, about 30 to about 50 vol-% stillage, or about 40 vol-% stillage.

In the conventional process employing gelatinization and liquefaction, stillage provides nutrients for efficient yeast fermentation, especially free amino nitrogen (FAN) required by yeast. The present invention can provide effective fermentation with reduced levels of stillage and even without added stillage. In an embodiment, the present method employs a preparation of plant material (e.g., fractionated plant material) that supplies sufficient quantity and quality of nitrogen for efficient fermentation under high gravity conditions (e.g., in the presence of high levels of reduced plant material). Thus, in an embodiment, no or only low levels of stillage can suffice.

However, the present method provides the flexibility to employ high levels of stillage if desired. The present method does not employ conventional liquefaction. Conventional liquefaction increases viscosity of the fermentation mixture and the resulting stillage. The present method produces lower viscosity stillage. Therefore, in an embodiment, increased levels of stillage can be employed in the present method without detrimental increases in viscosity of the fermentation mixture or resulting stillage.

Further, although not limiting to the present invention, it is believed that conventional saccharification and fermentation processes require added FAN due to undesirable “Maillard Reactions” which occur during high temperature gelatinization and liquefaction. The Maillard Reactions consume FAN during cooking. As a result, the conventional process requires adding stillage (or another source of FAN) to increase levels of FAN in fermentation. It is believed that the present process avoids temperature induced Maillard Reactions and provides increased levels of FAN in the reduced plant material, which are effectively utilized by the yeast in fermentation.

Saccharification can employ any of a variety of known enzyme sources (e.g., a microorganism) or compositions to produce fermentable sugars from the reduced plant material (e.g., fractionated plant material). In an embodiment, the saccharifying enzyme composition includes an amylase, such as an alpha amylase (e.g., an acid fungal amylase) or a glucoamylase.

In an embodiment, saccharification is conducted at a pH of about 6.0 or less, pH of about 3.0 to about 6.0, about 3.5 to about 6.0, about 4.0 to about 5.0, about 4.0 to about 4.5, about 4.5 to about 5.0, or about 4.5 to about 4.8. The initial pH of the saccharification mixture can be adjusted by addition of, for example, salt, NAOH, ammonia, sulfuric acid, phosphoric acid, process waters (e.g., stillage (backset), evaporator condensate (distillate), side stripper bottoms, and the like), and the like. Activity of certain saccharifying enzyme compositions (e.g., one including acid fungal amylase) can be enhanced at pH lower than the above ranges.

In an embodiment, saccharification is conducted at a temperature of about 25 to about 40 degrees C. or about 30 to about 35 degrees C.

In an embodiment, saccharifying can be carried out employing quantities of saccharifying enzyme composition selected to maintain low concentrations of dextrin in the fermentation broth. For example, the present process can employ quantities of saccharifying enzyme composition selected to maintain maltotriose (DP3) at levels at or below about 0.2 wt-% or at or below about 0.1 wt-%. For example, the present process can employ quantities of saccharifying enzyme composition selected to maintain dextrin with a degree of polymerization of 4 or more (DP4+) at levels at or below about 1 wt-% or at or below about 0.5 wt-%.

In an embodiment, saccharifying can be carried out employing quantities of saccharifying enzyme composition selected to maintain low concentrations of maltose in the fermentation broth. For example, the present process can employ quantities of saccharifying enzyme composition selected to maintain maltose at levels at or below about 0.3 wt-%. For maintaining low levels of maltose, suitable levels of acid fungal amylase and glucoamylase include about 0.05 to about 3 AFAU/gram dry solids reduced plant material (e.g., DSC) of acid fungal amylase and about 1 to about 2.5 (e.g., 2.4) AGU per gram dry solids reduced plant material (e.g., DSC) of glucoamylase. In an embodiment, the reaction mixture includes about 0.1 to about 2 AFAU/gram dry solids reduced plant material (e.g., DSC) of acid fungal amylase and about 1 to about 2.5 AGU per gram dry solids reduced plant material (e.g., DSC) of glucoamylase. In an embodiment, the reaction mixture includes about 0.3 to about 2 AFAU/gram dry solids reduced plant material (e.g., DSC) of acid fungal amylase and about 1 to about 2.5 AGU per gram dry solids reduced plant material (e.g., DSC) of glucoamylase. In an embodiment, the reaction mixture includes about 1 to about 2 AFAU/gram dry solids reduced plant material (e.g., DSC) of acid fungal amylase and about 1 to about 1.5 AGU per gram dry solids reduced plant material (e.g., DSC) of glucoamylase.

Glucoamylase

In certain embodiments, the present method can employ a glucoamylase. Glucoamylase is also known as amyloglucosidase and has the systematic name 1,4-alpha-D-glucan glucohydrolase (E.C. 3.2.1.3). Glucoamylase refers to an enzyme that removes successive glucose units from the non-reducing ends of starch. For example, certain glucoamylases can hydrolyze both the linear and branched glucosidic linkages of starch, amylose, and amylopectin. A variety of suitable glucoamylases are known and commercially available. For example, suppliers such as Novozymes and Genencor provide glucoamylases. The glucoamylase can be of fungal origin.

The amount of glucoamylase employed in the present process can vary according to the enzymatic activity of the amylase preparation. Suitable amounts include about 0.05 to about 6.0 glucoamylase units (AGU) per gram dry solids reduced plant material (e.g., DSC). In an embodiment, the reaction mixture can include about 1 to about 6 AGU per gram dry solids reduced plant material (e.g., DSC). In an embodiment, the reaction mixture can include about 1 to about 3 AGU per gram dry solids reduced plant material (e.g., DSC). In an embodiment, the reaction mixture can include about 1 to about 2.5 (e.g., 2.4) AGU per gram dry solids reduced plant material (e.g., DSC). In an embodiment, the reaction mixture can include about 1 to about 2 AGU per gram dry solids reduced plant material (e.g., DSC). In an embodiment, the reaction mixture can include about 1 to about 1.5 AGU per gram dry solids reduced plant material (e.g., DSC). In an embodiment, the reaction mixture can include about 1.2 to about 1.5 AGU per gram dry solids reduced plant material (e.g., DSC).

Acid Fungal Amylase

In certain embodiments, the present method employs an .alpha.-amylase. The .alpha.-amylase can be one produced by fungi. The .alpha.-amylase can be one characterized by its ability to hydrolyze carbohydrates under acidic conditions. An amylase produced by fungi and able to hydrolyze carbohydrates under acidic conditions is referred to herein as acid fungal amylase, and is also known as an acid stable fungal .alpha.-amylase. Acid fungal amylase can catalyze the hydrolysis of partially hydrolyzed starch and large oligosaccharides to sugars such as glucose. The acid fungal amylase that can be employed in the present process can be characterized by its ability to aid the hydrolysis of raw or native starch, enhancing the saccharification provided by glucoamylase. In an embodiment, the acid fungal amylase produces more maltose than conventional (e.g., bacterial) OL-amylases.

Suitable acid fungal amylase can be isolated from any of a variety of fungal species, including Aspergillus, Rhizopus, Mucor, Candida, Coriolus, Endothia, Enthomophtora, Irpex, Penicillium, Sclerotium and Torulopsis species. In an embodiment, the acid fungal amylase is thermally stable and is isolated from Aspergillus species, such as A. niger, A. saitoi or A. oryzae, from Mucor species such as M. pusillus or M. miehei, or from Endothia species such as E. parasitica. In an embodiment, the acid fungal amylase is isolated from Aspergillus niger. The acid fungal amylase activity can be supplied as an activity in a glucoamylase preparation, or it can be added as a separate enzyme. A suitable acid fungal amylase can be obtained from Novozymes, for example in combination with glucoamylase.

The amount of acid fungal amylase employed in the present process can vary according to the enzymatic activity of the amylase preparation. Suitable amounts include about 0.1 to about 10 acid fungal amylase units (AFAU) per gram of dry solids reduced plant material (e.g., dry solids corn (DSC)). In an embodiment, the reaction mixture can include about 0.05 to about 3 AFAU/gram dry solids reduced plant material (e.g., DSC). In an embodiment, the reaction mixture can include about 0.1 to about 3 AFAU/gram dry solids reduced plant material (e.g., DSC). In an embodiment, the reaction mixture can include about 0.3 to about 3 AFAU/gram dry solids reduced plant material (e.g., DSC). In an embodiment, the reaction mixture can include about 1 to about 2 AFAU/gram dry solids reduced plant material (e.g., DSC).

Fermenting

The present process includes fermenting sugars from reduced plant material (e.g., fractionated plant material) to ethanol. Fermenting can be effected by a microorganism, such as yeast. The fermentation mixture need not, and in an embodiment does not, include protease. However, the process waters may contain protease. The amount of protease can be less than that used in the conventional process. According to the present invention, fermenting is conducted on a starch composition that has not been cooked. In an embodiment, the present fermentation process produces potable alcohol. Potable alcohol has only acceptable, nontoxic levels of other alcohols, such as fusel oils. Fermenting can include contacting a mixture including sugars from the reduced plant material (e.g., fractionated plant material) with yeast under conditions suitable for growth of the yeast and production of ethanol. In an embodiment, fermenting employs the saccharification mixture.

Any of a variety of yeasts can be employed as the yeast starter in the present process. Suitable yeasts include any of a variety of commercially available yeasts, such as commercial strains of Saccharomyces cerevisiae. Suitable strains include “Fali” (Fleischmann's), Thermosac (Alltech), Ethanol Red (LeSafre), BioFerm AFT (North American Bioproducts), and the like. In an embodiment, the yeast is selected to provide rapid growth and fermentation rates in the presence of high temperature and high ethanol levels. In an embodiment, Fali yeast has been found to provide good performance as measured by final alcohol content of greater than 17% by volume.

The amount of yeast starter employed is selected to effectively produce a commercially significant quantity of ethanol in a suitable time, e.g., less than 75 hours.

Yeast can be added to the fermentation by any of a variety of methods known for adding yeast to fermentation processes. For example, yeast starter can be added as a dry batch, or by conditioning/propagating-. In an embodiment, yeast starter is added as a single inoculation. In an embodiment, yeast is added to the fermentation during the fermenter fill at a rate of 5 to 100 pounds of active dry yeast (ADY) per 100,000 gallons of fermentation mash. In an embodiment, the yeast can be acclimated or conditioned by incubating about 5 to 50 pounds of ADY per 10,000 gallon volume of fermenter volume in a prefermenter or propagation tank. Incubation can be from 8 to 16 hours during the propagation stage, which is also aerated to encourage yeast growth. The prefermenter used to inoculate the main fermenter can be from 1 to 10% by volume capacity of the main fermenter, for example, from 2.5 to 5% by volume capacity relative to the main fermenter.

In an embodiment, the fermentation is conducted at a pH of about 6 or less, pH of about 3 to about 6, about 3 to about 4.5, about 3.5 to about 6, about 4 to about 5, about 4 to about 4.5, about 4.5 to about 5, or about 4.5 to about 4.8. The initial pH of the fermentation mixture can be adjusted by addition of, for example, ammonia, sulfuric acid, phosphoric acid, process waters (e.g., stillage (backset), evaporator condensate (distillate), side stripper bottoms, and the like), and the like.

Although not limiting to the present invention, it is believed that known distillery yeast grow well over the pH range of 3 to 6, but are more tolerant of lower pH's down to 3.0 than most contaminant bacterial strains. Contaminating lactic and acetic acid bacteria grow best at pH of 5.0 and above. Thus, in the pH range of 3.0 to 4.5, it is believed that ethanol fermentation will predominate because yeast will grow better than contaminating bacteria.

In an embodiment, the present method can include varying the pH. It is believed that varying the pH can be conducted to reduce the likelihood of contamination early in fermentation and/or to increase yeast growth and fermentation during the latter stages of fermentation. For example, fermentation can include filling the fermenter at pH of about 3 to about 4.5 during the first half of fill. Fermentation can include increasing the slurry pH to pH of about 4.5 to about 6 during the second half of the fermenter fill cycle. Fermentation can include maintaining pH by adding fresh substrate slurry at the desired pH as described above. In an embodiment, during fermentation (after filling), pH is not adjusted. Rather, in this embodiment, the pH is determined by the pH of the components during filling.

In an embodiment, the pH is decreased to about five (5) or below in the corn process waters. In an embodiment, the pH is about pH 4 (e.g. 4.1) at the start of fermentation fill and is increased to about pH 5 (e.g. 5.2) toward the end of fermentation fill. In an embodiment, the method includes stopping pH control of the mash slurry after the yeast culture becomes established during the initial process of filling the fermenter, and then allowing the pH to drift up in the corn process waters during the end stages of filling the fermenter.

In an embodiment, fermentation is conducted for about to 25 (e.g., 24) to about to 150 hours, about 25 (e.g., 24) to about 96 hours, about 40 to about 96 hours, about 45 (e.g., 44) to about 96 hours, about 48 (e.g., 47) to about 96 hours. For example, fermentation can be conducted for about 30, about 40, about 50, about 60, or about 70 hours. For example, fermentation can be conducted for about 35, about 45, about 55, about 65, or about 75 hours.

In an embodiment, fermentation is conducted at a temperature of about 25 to about 40 degrees C. or about 30 to about 35 degrees C. In an embodiment, during fermentation the temperature is decreased from about 40 degrees C. to about 30 degrees C. or about 25 degrees C., or from about 35 degrees C. to about 30 degrees C., during the first half of the fermentation, and the temperature is held at the lower temperature for the second half of the fermentation. In an embodiment, the temperature can be decreased as ethanol is produced. For example, in an embodiment, during fermentation the temperature can be as high as about 99 degrees F. and then reduced to about 79 degrees F. This temperature reduction can be coordinated with increased ethanol titers (%) in the fermenter.

In an embodiment, the present method includes solids staging. Solids staging includes filling at a disproportionately higher level of solids during the initial phase of the fermenter fill cycle to increase initial fermentation rates. The solids concentration of the mash entering the fermenter can then be decreased as ethanol titers increase and/or as the fermenter fill cycle nears completion. In an embodiment, the solids concentration can be about 40% (e.g. 41%) during the first half of the fermentation fill. This can be decreased to about 25% after the fermenter is 50% full and continuing until the fermenter fill cycle is concluded. In the above example, such a strategy results in a full fermenter with solids at 33%.

It is believed that solids staging can accelerate enzyme hydrolysis rates and encourage a rapid onset to fermentation by using higher initial fill solids. It is believed that lowering solids in the last half of fill can reduce osmotic pressure related stress effects on the yeast. By maintaining overall fermenter fill solids within a specified range of fermentability, solids staging improves the capacity of the yeast to ferment high gravity mashes toward the end of fermentation.

Simultaneous Saccharification and Fermentation

The present process can include simultaneously converting reduced plant material (e.g., fractionated plant material) to sugars and fermenting those sugars with a microorganism such as yeast. Simultaneous saccharifying and fermenting can be conducted using the reagents and conditions described above for saccharifying and fermenting.

In an embodiment, saccharification and fermentation is conducted at a temperature of about 25 to about 40 degrees C. or about 30 to about 35 degrees C. In an embodiment, during saccharification and fermentation the temperature is decreased from about 40 to about 25 degrees C. or from about 35 to about 30 degrees C. during the first half of the saccharification, and the temperature is held at the lower temperature for the second half of the saccharification.

Although not limiting to the present invention, it is believed that higher temperatures early during saccharification and fermentation can increase conversion of starch to fermentable sugar when ethanol concentrations are low. This can aid in increasing ethanol yield. At higher ethanol concentrations, this alcohol can adversely affect the yeast. Thus, it is believed that lower temperatures later during saccharification and fermentation are beneficial to decrease stress on the yeast. This can aid in increasing ethanol yield.

Also not limiting to the present invention, it is believed that higher temperatures early during saccharification and fermentation can reduce viscosity during at least a portion of the fermentation. This can aid in temperature control. It is also believed that lower temperatures later during saccharification and fermentation are beneficial to reduce the formation of glucose after the yeast has stopped fermenting. Glucose formation late in fermentation can be detrimental to the color of the distillers dried residue co-product.

In an embodiment, saccharification and fermentation is conducted at a pH of about 6 or less, pH of about 3 to about 6, about 3.5 to about 6, about 4 to about 5, about 4 to about 4.5, about 4.5 to about 5, or about 4.5 to about 4.8. The initial pH of the saccharification and fermentation mixture can be adjusted by addition of, for example, ammonia, sulfuric acid, phosphoric acid, process waters (e.g., stillage (backset), evaporator condensate (distillate), side stripper bottoms, and the like), and the like.

In an embodiment, saccharification and fermentation is conducted for about to 25 (e.g., 24) to about to 150 hours, about 25 (e.g., 24) to about 72 hours, about 45 to about 55 hours, about 50 (e.g., 48) to about 96 hours, about 50 to about 75 hours, or about 60 to about 70 hours. For example, saccharification and fermentation can be conducted for about 30, about 40, about 50, about 60, or about 70 hours. For example, saccharification and fermentation can be conducted for about 35, about 45, about 55, about 65, or about 75 hours.

In an embodiment, simultaneous saccharifying and fermenting can be carried out employing quantities of enzyme and yeast selected to maintain high concentrations of yeast and high levels of budding of the yeast in the fermentation broth. For example, the present process can employ quantities of enzyme and yeast selected to maintain yeast at or above about 200 cells/mL, at or above about 300 cells/mL, or at about 300 to about 600 cells/mL.

In an embodiment, simultaneous saccharifying and fermenting can be carried out employing quantities of enzyme and yeast selected for effective fermentation without added exogenous nitrogen; without added protease; and/or without added backset. Backset can be added, if desired, to consume process water and reduce the amount of wastewater produced by the process. In addition, the present process maintains low viscosity during saccharifying and fermenting.

In an embodiment, simultaneous saccharifying and fermenting can be carried out employing quantities of enzyme and yeast selected to maintain low concentrations of soluble sugar in the fermentation broth. In an embodiment, simultaneous saccharifying and fermenting can be carried out employing quantities of enzyme and yeast selected to maintain low concentrations of glucose in the fermentation broth. For example, the present process can employ quantities of enzyme and yeast selected to maintain glucose at levels at or below about 2 wt-%, at or below about 1 wt-%, at or below about 0.5 wt-%, or at or below about 0.1 wt-%. For example, the present process can employ quantities of enzyme and yeast selected to maintain glucose at levels at or below about 2 wt-% during saccharifying and fermenting. For example, the present process can employ quantities of enzyme and yeast selected to maintain glucose at levels at or below about 2 wt-% from hours 0-10 (or from 0 to about 15% of the time) of saccharifying and fermenting. For example, the present process can employ quantities of enzyme and yeast selected to maintain glucose at levels at or below about 1 wt-%, at or below about 0.5 wt-%, or at or below about 0.1 wt-% from hours 12-54 (or from about 15% to about 80% of the time) of saccharifying and fermenting. For example, the present process can employ quantities of enzyme and yeast selected to maintain glucose at levels at or below about 1 wt-% from hours 54-66 (or about from 80% to about 100% of the time) of saccharifying and fermenting.

In an embodiment, simultaneous saccharifying and fermenting can be carried out employing quantities of enzyme and yeast selected to maintain low concentrations of maltose (DP2) in the fermentation broth. For example, the present process can employ quantities of enzyme and yeast selected to maintain maltose at levels at or below about 0.5 wt-% or at or below about 0.2 wt-%.

In an embodiment, simultaneous saccharifying and fermenting can be carried out employing quantities of enzyme and yeast selected to maintain low concentrations of dextrin in the fermentation broth. For example, the present process can employ quantities of enzyme and yeast selected to maintain maltotriose (DP3) at levels at or below about 0.5 wt-%, at or below about 0.2 wt-%, or at or below about 0.1 wt-%. For example, the present process can employ quantities of enzyme and yeast selected to maintain dextrin with a degree of polymerization of 4 or more (DP4+) at levels at or below about 1 wt-% or at or below about 0.5 wt-%.

In an embodiment, simultaneous saccharifying and fermenting can be carried out employing quantities of enzyme and yeast selected to maintain low concentrations of fusel oils in the fermentation broth. For example, the present process can employ quantities of enzyme and yeast selected to maintain fusel oils at levels at or below about 0.4 to about 0.5 wt-%.

For example, simultaneous saccharifying and fermenting can employ acid fungal amylase at about 0.05 to about 10 AFAU per gram of dry solids reduced plant material (e.g., DSC) and glucoamylase at about 0.5 to about 6 AGU per gram dry solids reduced plant material (e.g., DSC). For example, simultaneous saccharifying and fermenting can employ acid fungal amylase at about 0.1 to about 10 AFAU per gram of dry solids reduced plant material (e.g., DSC) and glucoamylase at about 0.5 to about 6 AGU per gram dry solids reduced plant material (e.g., DSC). For example, simultaneous saccharifying and fermenting can employ acid fungal amylase at about 0.3 to about 3 AFAU per gram of dry solids reduced plant material (e.g., DSC) and glucoamylase at about 1 to about 3 AGU per gram dry solids reduced plant material (e.g., DSC). For example, simultaneous saccharifying and fermenting can employ acid fungal amylase at about 1 to about 2 AFAU per gram of dry solids reduced plant material (e.g., DSC) and glucoamylase at about 1 to about 1.5 AGU per gram dry solids reduced plant material (e.g., DSC).

Additional Ingredients for Saccharification and/or Fermentation

The saccharification and/or fermentation mixture can include additional ingredients to increase the effectiveness of the process. For example, the mixture can include added nutrients (e.g., yeast micronutrients), antibiotics, salts, added enzymes, and the like. Nutrients can be derived from stillage or backset added to the liquid. Suitable salts can include zinc or magnesium salts, such as zinc sulfate, magnesium sulfate, and the like. Suitable added enzymes include those added to conventional processes, such as protease, phytase, cellulase, hemicellulase, exo- and endo-glucanase, xylanase, and the like.

Burn-Out of Residual Starches for Subsequent Secondary Fermentation

In an embodiment, the present method can include heat treatment of the stillage. This heat treatment can convert starches to dextrins and sugars for subsequent fermentation in a process known as burn-out. Such a treatment step can also reduce fouling of distillation trays and evaporator heat exchange surfaces. In an embodiment, heat treatment staging can be performed on whole stillage or thin stillage. Following enzymatic treatment of the residual starches, in an embodiment, the resulting dextrins and sugars can be fermented within the main fermentation process as recycled backset or processed in a separate fermentation train to produce ethanol. In an embodiment, the liquefaction and saccharification on whole stillage or thin stillage produced by centrifugation can be accelerated after distillation.

Fractionation of Solids from Fermentation

Large pieces of germ and fiber can ferment the residual starch in the fermenter. After fermentation, the fractions could be removed prior to or after distillation. Removal can be effected with a surface skimmer before to distillation. In an embodiment, screening can be performed on the mixture. The screened material can then be separated from the ethanol/water mix by, for example, centrifugation and rotary steam drum drying, which can remove the residual ethanol from the cake. In embodiments in which the larger fiber and germ pieces are removed prior to bulk mixture distillation, a separate stripper column for the fiber/germ stream can be utilized. Alternatively, fiber and germ could be removed by screening the whole stillage after distillation.

In an embodiment, all the components are blended and dried together. The fiber and germ can be removed from the finished product by aspiration and/or size classification. The fiber from the DDGS can be aspirated. Removal of fiber by aspiration after drying can increase the amount of oil and protein in the residual DDGS, for example, by 0.2 to 1.9% and 0.4 to 1.4%, respectively. The amount of NDF in the residual DDGS can decrease, for example, by 0.1 to 2.8%.

In an embodiment, fractionation can employ the larger fiber and germ pieces to increase the particle size of that part of the DDGS derived from the endosperm, as well as to improve syrup carrying capacity. A ring dryer disintegrator can provide some particle size reduction and homogenization.

Methods and Systems for Drying Wet Cake to Make Distillation Dried Residues

The mixture produced by fermentation includes ethanol, other liquids, and solid material. Centrifugation and/or distillation of the mixture can yield solids known as wet cake and liquids known as thin stillage. The wet cake can be dried to produce distillation dried residue. The thin stillage can be concentrated to a syrup, which can be added to the wet cake or distillation dried residue and the mixture then dried to form distillation dried residue plus solubles. The present method can include drying the wet cake to produce distillation dried residue. The present method can include drying the syrup plus distillation dried residue to produce distillation dried residue plus solubles. The distillation dried residue can be produced from whole pulses (e.g., lentils) or from fractionated pulses. The present method can produce high protein distillation dried residue and/or distillation dried residue with improved physical characteristics. Such distillation dried residues are described below.

Conventional ethanol production processes employed drum dryers. Advantageously, in an embodiment, the present method and system can employ a flash or ring dryer. Flash or ring dryers have not previously been employed in processes like the present one. Configurations of flash and ring dryers are known. Briefly, a flash or ring dryer can include a vertical column through which a pre-heated air stream moves the wet cake. For example, a flash or ring dryer can include one or more inlets that provide entry of heat or heated air into the dryer. This dries the wet cake. The dried wet cake is transported to the top of a column. In a ring dryer, further drying can be accomplished by moving the wet cake through one or more rings connected to the column. For example, a ring dryer can include one or more inlets through which heated air enters a ring structure which propels or circulates the wet cake in or around the ring structure. The dried wet cake can then be pneumatically conveyed to down-stream separating equipment such as a cyclone or dust collector.

The present method can include employing a flash dryer to dry (i.e., flash drying) the wet cake and to produce distillation dried residue. The present method can include employing a flash dryer to dry (i.e., flash drying) the syrup plus distillation dried residue to produce distillation dried residue plus solubles. Employing a flash dryer can produce high protein distillation dried residue and/or distillation dried residue with improved physical characteristics. Such distillation dried residues are described below.

The present method can include employing a ring dryer to dry (i.e., ring drying) the wet cake and to produce distillation dried residue. The present method can include employing a ring dryer (i.e., ring drying) to dry the syrup plus distillation dried residue to produce distillation dried residue plus solubles. Employing a ring dryer can produce high protein distillation dried residue and/or distillation dried residue with improved physical characteristics. Such distillation dried residues are described below.

The present method can include employing a fluid bed dryer to dry (i.e., fluid bed drying) the wet cake and to produce distillation dried residue. The present method can include employing a fluid bed dryer to dry (i.e., fluid bed drying) the syrup plus distillation dried residue to produce distillation dried residue plus solubles. Employing a fluid bed dryer can produce high protein distillation dried residue and/or distillation dried residue with improved physical characteristics. Such distillation dried residues are described below.

The present method can include adding syrup (backset or thin stillage) to the wet cake before, during, or after drying. In an embodiment, the present method includes adding syrup (backset or thin stillage) to the wet cake during drying. For example, the method can include mixing wet cake and syrup in the dryer. For example, the method can include flowing or injecting syrup into the flash, ring, or fluid bed dryer. In an embodiment, the present method includes adding syrup into the column or ring of the dryer in the presence of wet cake and/or distillation dried residue.

Although not limiting to the present invention, it is believed that flash and/or ring dryers differ from rotary or drum dryers by providing decreased exposure of wet cake to high temperatures of the drying process. A rotary or drum dryer generally has high temperature metal that is in prolonged contact with the wet cake product. It is believed that prolonged contact of this high temperature metal with the wet cake can result in browned, burned, or denatured distillation dried residues or distillation dried residues plus solubles. Further, the internal air temperature can be higher in a rotary or drum dryer.

Accordingly, in an embodiment, the present method can include drying the wet cake or wet cake plus syrup for a shorter time than employed with a rotary or drum dryer, and obtaining distillation dried residue or distillation dried residue plus solubles that has been sufficiently dried. Accordingly, in an embodiment, the present method can include drying the wet cake or wet cake plus syrup at a lower temperature than employed with a rotary or drum dryer, and obtaining distillation dried residue or distillation dried residue plus solubles that has been sufficiently dried. In an embodiment, the method includes changing the drying temperature during drying.

Although not limiting to the present invention, in certain embodiments, such drying systems and methods can provide one or more advantages such as decreased energy consumption in drying, decreased leakage from the drying system.

An embodiment of this invention is the use of flash or ring dryer(s) to change the conditions inside the dryer system to increase or decrease temperature. An embodiment of this invention is the use of flash or ring dryer(s) to change the conditions inside the dryer system to increase or decrease the moisture. An embodiment of this invention is the use of flash or ring dryer(s) to change the conditions inside the dryer system to increase or decrease recycle speed. An embodiment of this invention is the use of flash or ring dryer(s) to change the conditions inside the dryer system to increase or decrease the feed rate into the dryer system.

Continuous Fermentation

The present process can be run via a batch or continuous process. A continuous process includes moving (pumping) the saccharifying and/or fermenting mixtures through a series of vessels (e.g., tanks) to provide a sufficient duration for the process. For example, a multiple stage fermentation system can be employed for a continuous process with 48-96 hours residence time. For example, reduced plant material (e.g., fractionated plant material) can be fed into the top of a first vessel for saccharifying and fermenting. Partially incubated and fermented mixture can then be drawn out of the bottom of the first vessel and fed in to the top of a second vessel, and so on.

Although not limiting to the present invention, it is believed that the present method is more suitable than conventional methods for running as a continuous process. It is believed that the present process provides reduced opportunity for growth of contaminating organisms in a continuous process. At present, the majority of dry grind ethanol facilities employ batch fermentation technology. This is in part due to the difficulty of preventing losses due to contamination in these conventional processes. For efficient continuous fermentation using traditional liquefaction technology, the conventional belief is that a separate saccharification stage prior to fermentation is necessary to pre-saccharify the mash for fermentation. Such pre-saccharification insures that there is adequate fermentable glucose for the continuous fermentation process.

The present method achieves efficient production of high concentrations of ethanol without a liquefaction or saccharification stage prior to fermentation. This is surprising since this conventional wisdom teaches that it is necessary to have adequate levels of fermentable sugar available during the fermentation process when practiced in a continuous mode. In contrast the present method can provide low concentrations of glucose and efficient fermentation. In the present method, it appears that the glucose is consumed rapidly by the fermenting yeast cell. It is believed that such low glucose levels reduce stress on the yeast, such as stress caused by osmotic inhibition and bacterial contamination pressures. According to the present invention, ethanol levels greater than 18% by volume can be achieved in about 45 to about 96 hours.

Fermentation with Continuous Substrate Addition and Continuous Clarification

In an embodiment, ethanol production can take place in a single fermenter that can be adapted to discharge a portion of its contents to a centrifuge operating in a continuous mode. The centrifuge operating in a continuous mode can be capable of recovering unfermented substrate and yeast cells as centrifuge cake, which can be recycled to the fermenter. The clarified centrate, which is depleted of fermentable carbohydrate or fermenting organism, can be sent to a stripper column for recovery of ethanol. In an embodiment, the liquids can be cooled and recycled to the fermenter as process water to dilute and maintain fermenter ethanol content at approximately ten percent (10%).

As substrate is continuously converted to alcohol and recovered in distillation, the method can include adding substrate to the ongoing fermentation. Such a system can operate on a continuous basis, maintaining “isoethanol” conditions by balancing the rate of adding fermentable substrate with the rate at which substrate is converted to ethanol and removed by the continuous distillation process. Adding fresh substrate can be accomplished by metering into the fermenter. The rate of adding fresh substrate can depend on the rate of fermentation, the quantity and quality of unfermented material recycled in the centrifuge cake, and the quantity and quality of process water recycled after distillation of the centrate fraction.

In an embodiment, the system operates at a high rate of ethanol productivity achievable by maintaining low alcohol content in the fermentation, high cell counts of fermenting yeast cells, and high fermentable substrate concentration. Saccharifying enzyme (e.g., glucoamylase) activity can be maintained by the recycle of enzyme with the cake fraction to maintain enzyme levels. Since the method of the present invention maintains low levels of carbohydrate throughout fermentation, even with cereal derived substrates, it is possible to distill off a fraction of the alcohol without unacceptably reducing fermentable sugar. In the process of the present invention unfermented substrate remains in the form of insoluble solids and is recovered and recycled in the centrifuge cake stream and is not exposed to the thermal degradation conditions present in distillation.

The present invention can use any of a variety of methods of clarifying. In an embodiment, clarifying can employ a membrane system, which can contain unfermented starches or enzymes and allow release or separation of a fraction containing ethanol. In an embodiment, clarifying can employ a membrane system, which can contain unfermented starches and enzymes and allow release or separation of a fraction containing ethanol. In an embodiment, clarifying can employ a membrane bioreactor system, which can contain unfermented starches and enzymes and allow release or separation of a fraction containing ethanol. In an embodiment, clarifying can employ a membrane bioreactor system, which can contain unfermented starches and enzymes and allow release or separation of a fraction containing ethanol. The membrane or membrane bioreactor system can allow the clarified ethanol portion to be directed to a stripper column for recovery of ethanol while unfermented substrate and yeast cells can recycle to the fermenter.

In an embodiment, the present invention can produce a total cumulative greater than about 9 vol-% (e.g. 9.9) ethanol in about 12 hours, about 17 vol-% (e.g. 17.7) ethanol in about 18 hours, about 27 vol-% (e.g. 27.4) ethanol in about 24 hours, about 33 vol-% (e.g. 34.0) ethanol in about 30 hours, about 38 vol-% (e.g. 39.3) ethanol in about 36 hours, 40 vol-% ethanol in about 40 hours, about 42 vol-% (e.g. 43.0) ethanol in about 42 hours; about 43 vol-% (e.g. 44.9) ethanol in about 48 hours; about 44 vol-% (e.g. 45.4) ethanol in about 60 hours; and about 44 vol-% (e.g. 45.4) ethanol in about 72 hours.

Endosperm, Fibers and Germ Fermentation

In an embodiment, the present process can ferment a portion of a reduced plant material, such as a pulse. For example, the process can ferment at least one of endosperm, fiber, or germ. The present process can increase ethanol production from such a portion of the pulse. In an embodiment, the present process can saccharify and ferment endosperm. Endosperm fermentation is lower in free amino nitrogen (FAN) towards the beginning of fermentation due to the removal of germ, which contains FAN. The present process can, for example, preserve the FAN quality of the endosperm compared to conventional high temperature liquefaction. An embodiment of the present invention includes the use of endosperm FAN, which can increase flexibility and efficiency of fermentation.

In an embodiment, the present process can employ endogenous enzyme activity in the residue. In an embodiment, dramatic increase in FAN in whole corn and defibered corn fermentations are reached compared to the initial mash slurry.

Conventional grain dry milling operations separate germ (containing oil) and bran or pericarp (fiber fraction) from the endosperm (starch and protein) portion of the pulse using a series of steps and procedures. These steps and procedures include: grain cleaning, tempering, degerming, particle size reduction, roller milling, aspirating, and sifting. This process differs from the traditional wet milling of grains (commonly corn) which are more expensive and water intensive, but capable of achieving cleaner separations of the components of the grain. Dry milling processes offer a version of separating components using lower capital costs for facilities. Also, these processes require less water for operation. The tempering process in dry milling requires less water than required in wet milling.

The competitiveness of dry pulse fractionation processes is enhanced when the process of the present invention is utilized for ethanol conversion of these fractions. Traditionally dry milling processes produce various grades of each fraction (germ, bran, and endosperm). In an embodiment, the present method provides bran and endosperm fractions that can be more readily fermented. Depending on the desired purity of each fraction, the fractions can either be pooled to create composites of each stream, or the fractions can be processed individually.

Yeast uses FAN in the present process. In the conventional liquefaction process, FAN levels fall throughout fermentation as yeast cells assimilate and metabolize available FAN during the course of fermentation. Toward the end of fermentation in the conventional process, FAN levels rise illustrating the liberation of cellular FAN coinciding with death and lysis of yeast cells. In contrast, FAN utilization kinetics in the raw starch process is more rapid. FAN levels reach a minimum at least 24 hours earlier, and then begin increasing dramatically. Some of the increase of FAN is due to yeast cell death resulting from the accelerated fermentation.

High Alcohol Mixture

The present invention also relates to a high alcohol mixture. In an embodiment, the process of the present invention produces a mixture containing greater than 18 vol-% ethanol. The present process can produce such a high alcohol mixture in about 40 to about 96 hours or about 45 to about 96 hours. In an embodiment, the mixture includes 18 vol-% to about 23 vol-% ethanol. For example, the present method can produce alcohol contents in the fermenter of 18 to 23% by volume in about 45 to 96 hours.

By way of further example, the present method can produce alcohol content in the fermenter of 18 to 23% by volume in about 45 to 96 hours. In certain embodiments, the majority of the alcohol (80% or more of the final concentration) is produced in the first 45 hours. Then, an additional 2 to 5 vol-% alcohol can be produced in the final 12-48 hours. Concentrations of ethanol up to 23 vol-% can be achieved with fermentation time up to 96 hours. It can be economically advantageous to harvest after 48 to 72 hours of fermentation to increase fermenter productivity.

The present mixture can include this high level of ethanol even when it includes high levels of residual starch. For example, the present mixture can include ethanol at 18 to 23 vol-% when it contains 0 to 30% residual starch. The present mixture can contain residual starches as low as 0% to as high as 20% residual starch.

By conventional measures, high levels of residual starch indicate inefficient fermentation, which yields only low levels of ethanol. In contrast, although not limiting to the present invention, it is believed that the present method results in fewer Maillard type reaction products and more efficient yeast fermentation (e.g., reduced levels of secondary metabolites). This is believed to be due to the low glucose levels and low temperatures of the present method compared to conventional saccharification and liquefaction. Thus, the present method can produce more alcohol even with higher levels of residual starch.

In an embodiment, the present mixture includes fewer residual byproducts than conventional mixtures, even though residual starch can be higher. For example, residual glucose, maltose, and higher dextrins (DP3+) can be as much as 0.8 wt-% lower than in conventional mixtures produced under similar fermentation conditions. By way of further example, residual glycerol can be as much as 0.7 wt-% less. Lactic acid and fusel oils can also be significantly reduced. For example, the present mixture can include less than or equal to about 0.2 wt-% glucose, about 0.4 wt-%, about 0.1 wt-% DP3, undetectable DP4+, 0.7 wt-% glycerol, about 0.01 wt-% lactic acid, and/or about 0.4 wt-% fusel oils.

High Protein Distillation Dried Residue

The present invention also relates to a distillation dried residue product. The distillation dried residue can also include elevated levels of one or more of protein, fat, fiber (e.g., neutral detergent fiber (NDF)), and starch. For example, the present distillation dried residue can include 34 or more wt-% protein, about 25 to about 60 wt-% protein, about 25 to about 50 wt-% protein, or about 30 to about 45 wt-% protein. In certain circumstances the amount of protein is about 1 to about 2 wt-% more protein than produced by the conventional process. For example, the distillation dried residue can include 15 or more wt-% fat, about 13 to about 17 wt-% fat, or about 1 to about 6 wt-% more fat than produced by the conventional process. For example, the distillation dried residue can include 31 or more wt-% fiber, about 23 to about 37 wt-% fiber, or about 3 to about 13 wt-% more fiber than produced by the conventional process. For example, the distillation dried residue can include 12 or more wt-% starch, about 1 to about 23 wt-% starch, or about 1 to about 18 wt-% more starch than produced by the conventional process.

In an embodiment, the present distillation dried residue includes elevated levels of B vitamins, vitamin C, vitamin E, folic acid, and/or vitamin A, compared to conventional distillation dried residue products. The present distillation dried residue has a richer gold color compared to conventional distillation dried residue products.

Distillation Dried Residue with Improved Physical Characteristics

The present invention also relates to a distillation dried residue with one or more improved physical characteristics, such as decreased caking or compaction or increased ability to flow. The present process can produce such an improved distillation dried residue.

Although not limiting to the present invention, it is believed that the present process can produce fermentation solids including higher molecular weight forms of carbohydrates. Such fermentation solids can, it is believed, exhibit a higher glass transition temperature (i.e. higher T_{g} values) compared to solids from the conventional process. For example, residual starches can have a high T.sub.g value. Thus, through control of starch content in the DDG and DDGS, the present process can manufacture DDG or DDGS with target T_{g} values.

Further, according to the present invention, adding an alkaline syrup blend (e.g., syrup plus added lime or other alkaline material) to the fermentation solids (e.g., distillation dried residues) can provide decreased caking or compaction or increase ability to flow to the distillation dried residue with solubles (DDGS).

Although not limiting to the present invention, it is believed that organic acids such as lactic, acetic, and succinic acids which are produced in fermentation have a lower T_{g} value than their corresponding calcium salts. Maintenance of residual carbohydrate in higher molecular weight form, or addition of lime to form calcium salts of organic acids, are two strategies for forming higher T_{g} value co-products that will be less likely to undergo the glass transition, resulting in the deleterious phenomenon known as caking.

In an embodiment, DDG or DDGS of or produced by the method of the present invention flows more readily than DDG or DDGS produced by the conventional process. Although not limiting to the present invention, it is believed that the example method need not destroy protein in the fermented plant material (e.g., fractionated plant material). Corn contains prolamins, such as zein. Grain sorghum, for example, contains a class of zein-like proteins known as kafirins, which resemble zein in amino acid composition. The thermal degradation that occurs during liquefaction, distillation, and high temperature drying produces DDG and DDGS including significant amounts of degraded protein. It is believed that the process of the present invention can provides improved levels of the prolamin fraction of pulses.

Extended exposure to high alcohol concentrations that can be achieved by the present process can condition the proteins in the plant material (e.g., fractionated plant material). This can solubilize some of the proteins. For example, it is believed that in distillation the ethanol concentration reaches levels that can solubilize prolamins. Upon the removal, or “stripping,” of ethanol from the mixture, prolamins can be recovered in concentrated form in DDG and DDGS. The resulting high protein content of DDG and DDGS can be advantageous for various end uses of DDG and DDGS, for example in further processing or compounding.

In an embodiment, the present method can operate on fractionated plant material (such as endosperm, fiber, other parts of pulses) to provide a protein enriched solid product from fermentation. For example, the present method operated on fractionated plant material can produce a DDG enriched in prolamin.

In an embodiment, the process of the present invention can provide DDG and DDGS with different, predetermined T_{g} values. The process of the present invention can ferment fractions containing high, medium, or low levels of zein, thus varying the glass transition temperature of the resulting DDG or DDGS. The resulting co-product T_{g} can be directly proportional to the prolamin protein content. The process of the current invention is desirable for the fermentation of high-protein pulses. This also allows production of DDG and DDGS with a higher prolamin content.

Residual starch remaining at the end of fermentation preferentially segregates into the thin stillage fraction, which is subsequently evaporated to produce syrup. The wet cake fraction produced by the present method, which can be dried separately to produce DDG, can be higher in prolamin protein (such as zein) than conventional DDG. The present process allows syrup and wet cake blend ratios to be varied. This results in DDG/DDGS with varying ratios of prolamin protein (such as zein) and residual starch. As the residual starch in the wet cake reduces the protein in the wet cake increases. This indicates an inverse relationship. A similar response occurs in the syrup fraction.

It is believed that starch can segregate into the liquid fraction. The amount of starch in the DDGS can be varied by blending syrup at rates ranging from 0 lbs. dry weight of syrup solids to 1.2 lbs. of syrup solids per lb. of wet cake solids before, and various times during drying to create the final DDGS product. The disproportionate segregation of residual starches into the backset or thin stillage fraction can provide both the aforementioned burn-out and secondary fermentation to be performed on these fractions. Since the thin stillage is evaporated to produce syrup, the centrifuge mass balance also enables DDGS production at various T_{g} values depending on the desired properties and their dependence on T_{g}.

Emissions

The present invention has emissions benefits. Emissions benefits result in the reduction in byproducts created in the ethanol manufacturing process. There is a marked reduction in extraction of fats and oils in the mash from the germ fraction of pulses. There is a reduction of byproducts from Maillard reactions typically formed during cooking and liquefaction. And there is a reduction in fermentation byproducts. These observations result in reduced emissions during the recovery of co-products. The concentration and emission rates of volatile organic compounds (VOC), carbon monoxide (CO), nitric oxide compounds (NOx), sulfur oxides (SO2), and other emissions are considerably lower. Note that other manufacturers have attempted to lower emissions by manufacturing wet cake instead of drying to DDG or DDGS.

1. A method, comprising:
fractionating a pulse into a fiber fraction, a protein fraction, and a carbohydrate fraction;
extracting the protein fraction from the other fractions;
processing at least the protein fraction into a food product; and
after extracting the protein fraction, processing at least the carbohydrate fraction into an alcohol biofuel.

2. The method of claim 1, wherein the pulse comprises one of a pea crop, a bean crop, or a lentil crop.

3. The method as in claim 1, wherein the alcohol biofuel comprises ethanol.

4. The method as in claim 1, wherein the food product is one of a extruded or baked cereal, textured protein, protein concentrate or a fish or animal feed.

5. The method as recited in claim 1, wherein processing at least the carbohydrate utilizes two enzymes.

6. The method as recited in claim 4, wherein one of the two enzymes comprises amylase.

7. A pulse-based ecosystem model, comprising:
growing a pulse crop;
extracting a protein portion from the pulse crop to make one of a flour, a cereal, a protein concentrate, a livestock feed, or a fish feed;
fermenting remaining fractions of the pulse crop to make one of an alcohol biofuel or a biofuel gas.

8. The pulse-based ecosystem model of claim 7, further comprising generating electricity from the alcohol biofuel or the biofuel gas; and
applying the electricity to at least extract the protein portion from the pulse crop.

9. The pulse-based ecosystem model of claim 7, further comprising using the alcohol biofuel or the biofuel gas in a vehicle used for planting or maintaining the pulse crop.

10. A method for fractionating a pulse crop, comprising:
preprocessing the pulse crop to extract an edible protein concentrate from the pulse crop; and
generating a biofuel from remaining fractions of the pulse crop.

11. The method of claim 10, wherein the preprocessing includes
dehulling the pulse crop using a tangential or other abrasive dehulling device to remove tannins and insoluble fibers;
grinding the dehulled pulse crop with a mill;
screening the milled pulse crop with a screen having 0.1-0.4 mm openings;
analyzing the pulse crop for moisture, protein content, and ash;
drying the pulse crop when the moisture content past a threshold.

12. The method as recited in claim 11, further comprising:
extracting a protein fraction of the pulse crop with a wet fractionation technique including:
applying an extraction solution comprising one of distilled water, a salt solution of approximately 0.5M NaCl, or an alkaline solution of approximately pH 9 1.0N NaOH.

13. The method of claim 12, further comprising enhancing protein quality by transferring protein concentrates and isolates through a
filter utilizing a micro porous membrane to remove bioactive compounds, protein anti-nutritional compounds, and immune-triggering proteins.

14. The method of claim 12, further comprising fractionating a protein concentrate or globulin isolate of the pulse crop to albumins and globulins by a dialysis method.
